(19) United States
(12) Patent Application Publication
(10) Pub. No.: US 2012/0178144 A1
(43) Pub. Date: Jul. 12, 2012

(54) METHOD OF PRODUCING CELLULASE

(76) Inventors: MOHD ZAHANGIR ALAM, Kuala Lumpur (MY); Mohd Ismail Abd Karim, Kuala Lumpur (MY); Suleyman Aremu Muyibi, Kuala Lumpur (MY); Hamzah Mohd Salleh, Kuala Lumpur (MY); Nassereldeen Ahmed Kabbashi, Kuala Lumpur (MY)

(21) Appl. No.: 13/345,160

(22) Filed: Jan. 6, 2012

(30) Foreign Application Priority Data
Jan. 7, 2011 (MY) PI 20110081

(51) Int. Cl. C12N 9/42

(52) U.S. Cl. 435/209

(57) ABSTRACT

A method is described for producing cellulase by fermentation wherein the fermentation is conducted using Trichoderma reesei in a medium containing sewage treatment plant sludge. By utilizing sewage sludge, wastewater could be managed effectively. This is an approach which not only reduces environmental pollution, but also produces cellulase effectively. After the fermentation process, the fermentation product is filtered in order to separate the produced cellulase from the medium that contains biomass. There are two filtration steps involved in this process. The produced cellulase is micro-filtrated to remove the fungal biomass. After that, the filtrate is further filtered by ultra-filtration to obtain the cellulase. The fungal biomass, including biosolids/debris, is considered as the potential raw material for compost/bio-fertilizer.

### FIELD OF INVENTION

The present invention relates to a method of producing cellulase. More particularly, the present invention relates to a method of producing cellulase by fermentation using Trichoderma reesei in a medium containing sewage treatment plant sludge.

### BACKGROUND OF THE INVENTION

Sewage, also known as wastewater flow, is water that carries waste away in drains from a community. As it contains hazardous and toxic materials as well as pathogenic organisms, sewage can be life threatening to human and animals as it can transmit diseases. Hence, management of sewage has always been one of the prime concerns around the world. The application of biological-based treatment to convert the sewage into an environmentally-safe fluid waste stream suitable for disposal or reuse is an effective and acceptable way to manage sewage. This process is called sewage treatment.

One of the final products of the process is Sewage Treatment Plant (STP) sludge which is a good source for microorganisms to grow as it is inexpensive and rich in nutrients. It is not only a solution for wastewater management, but also produces value added product. This could be done via fermentation by microorganisms. Fermentation is a metabolic process that breaks down complex organic compounds into simple components. Fermentation is carried out by using microorganisms whereby one of the most commonly used microorganisms is fungi. Cellulase can be produced as the value added product via fermentation by fungi in sewage treatment plant sludge.

There are several patents relating to the production of cellulase. Japanese Patent No. 02-119774 discloses a method in obtaining cellulase through fermentation by Trichoderma reesei in a medium containing finely crushed wood powder as carbon source.

Another method of producing cellulolytic enzyme is disclosed in Malaysian Patent No. 20064744. The process is conducted in a medium containing Palm Oil Mill Effluent (POME) by Trichoderma harzianum.

A process of producing cellulase by aerobic fermentation in a culture medium comprising Trichoderma reesei is described in U.S. Pat. No. 4,762,788. The medium further comprises an inorganic salt and a small quantity of cellulose and sugar.

There are a variety of ways to produce cellulase using different microorganisms and fermenting medium. However, simple and effective production methods with the use of materials that are easily obtained yet not costly are most desirable.

### SUMMARY OF THE INVENTION

The present invention shall utilize a medium containing sewage treatment plant sludge and Trichoderma reesei to produce cellulase. By utilizing sewage sludge, wastewater could be managed effectively. This is an approach which not only reduces environmental pollution but also produces cellulase effectively.

The primary aspect of the present invention is to produce cellulase via simple steps by Trichoderma reesei in a fermentation medium containing sewage treatment plant sludge.

Another aspect of the present invention is to produce cellulase by utilizing materials that are easy to obtain and not costly.

Still another aspect of the present invention is to produce cellulase in high yield under optimal conditions during fermentation.

Yet another aspect of the present invention is to produce cellulase via an approach that is beneficial to the environment.

At least one of the preceding objects is met, in whole or in part, by the present invention, in which the embodiment of the present invention describes a method of producing cellulase by fermentation wherein the fermentation is conducted using Trichoderma reesei in a medium containing sewage treatment plant sludge.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 is a diagram showing the production of cellulase by Trichoderma reesei in a medium containing sewage treatment plant sludge;

FIG. 2 is a graph showing the production of cellulase (U/ml) enzyme by fermentation using sewage treatment plant sludge as medium in a 300 L bioreactor;

FIG. 3 is a graph showing the pH of the fermentation medium during fermentation in a 300 L bioreactor;

FIG. 4 is a graph showing the total suspended solids (TSS) measurement in the fermentation medium during fermentation in the 300 L bioreactor;

FIG. 5 is a graph showing the dissolved oxygen concentration of the fermentation medium during fermentation in the 300 L bioreactor; and

FIG. 6 is a graph showing the filterability of the fermentation medium to evaluate the down streaming process in the 300 L bioreactor.

The same reference numerals refer to the same parts throughout the various Figures.

### DETAILED DESCRIPTION OF THE INVENTION

The following description of the preferred embodiment(s) is merely exemplary in nature and is in no way intended to limit the invention, or uses.

The present invention discloses a method of producing cellulase comprising the steps of performing fermentation using Trichoderma reesei in a medium containing sewage treatment plant (STP) sludge. Trichoderma reesei strains like RL-P37 and MCG-80 could also be used but Rut-C30 is the preferred Trichoderma reesei strain code to be applied in this fermentation process.

An inoculum of Trichoderma reesei is prepared to be applied in the fermentation process. Trichoderma reesei is cultured on Potato Dextrose Agar (PDA) plate in laminar flow for 3 to 7 days at a temperature of 28 to 32° C. After that, the culture is added with sterile distilled water and agitated at 130 to 180 rpm by shaking or stifling for 1 to 2 days. The suspended fungal cultures of Trichoderma reesei are filtered and the filtrate is used as inoculum.

Sewage treatment plant (STP) sludge is used as the basal medium for the fermentation process. Preferably, the STP sludge contains 0.5 to 1% (w/v) of total suspended solid (TSS). The fermenting medium also includes one or more of cellulose, sucrose, peptone, polysorbate 80, potassium dihydrogen phosphate and magnesium sulfate heptahydrate. The cellulose has concentration ranging from 0.5 to 2% (w/v), preferably at 1.5% (w/v) whereas the peptone has concentration ranging from 0.2 to 1.0% (w/v), preferably at 0.5% (w/v). On the other hand, concentration of the polysorbate 80 ranges from 0.1 to 0.5% (w/v). It is preferred that the concentration of polysorbate 80 is 0.2% (w/v). Sucrose has a concentration of 1.0% (w/v) or the concentration can also be within the range of 0.5 to 1.5% (w/v). Concentration of the potassium dihydrogen phosphate is 0.1 to 0.3% (w/v), preferably at 0.25% (w/v) while the magnesium sulfate heptahydrate has a concentration of 0.02 to 0.04% (w/v), preferably at 0.03% (w/v).

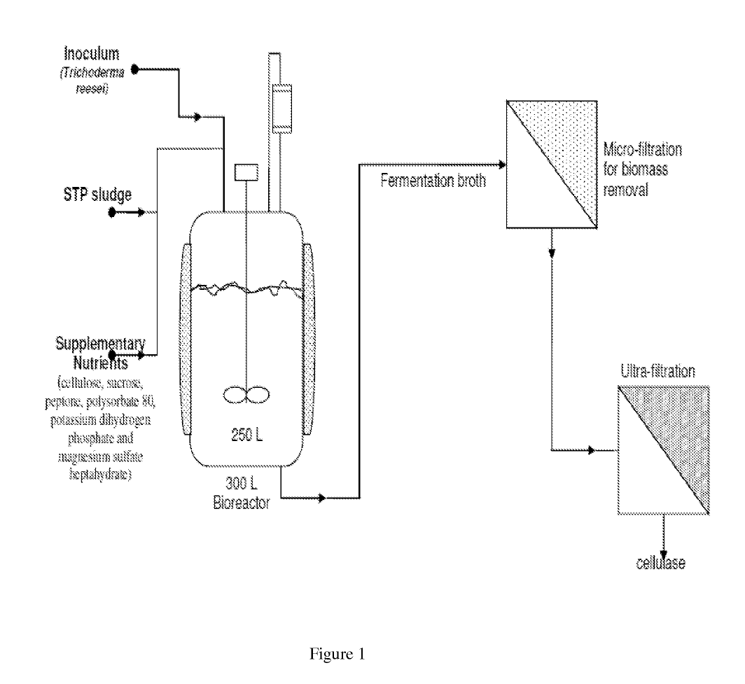

A variety of ways can be adopted to carry out the fermentation process. This includes shaking, stifling and static culture. Referring to FIG. 1, in a preferred embodiment, fermentation is performed in a bioreactor of 300 L with 250 L of the fermentation medium containing Trichoderma reesei in it. The medium is stirred at an agitation rate of 36 to 60 rpm, preferably at 36 rpm and aeration rate of 0.25 to 0.5 vvm, preferably at 0.3 vvm. Fermentation is carried out under optimum conditions. The pH of the fermentation medium is 5 at the start of the fermentation process. The pH of the medium changes depending on the fermentation reaction as the fermentation process is being carried out. Temperature of the medium is in the range of 31 to 33° C. The medium contains dissolved oxygen (DO) that has a concentration of more than 2 mg/L. Fermentation period is not specifically limited as it depends on the desired production amount of cellulolytic enzyme. However, fermentation period is preferred to be performed in 4-7 days.

After the fermentation process, the fermentation product is filtered in order to separate the produced cellulase from the medium that contains biomass. There are two filtration steps involved in this process. The produced cellulase is micro-filtrated to remove the fungal biomass. After that, the filtrate is further filtered by ultra-filtration to obtain the cellulase.

Although the description above contains many specifications, it is understood that the embodiment described herein is not intended as limitations on the scope of the invention.

### Example 1

The experiment to find out the optimum medium for the production of cellulase through fermentation is performed. Three major constituents which are cellulose, peptone and sucrose are applied with sewage treatment plant sludge as the fermentation medium for the growth of fungus. A maximum of about 17 U/mL of cellulase activity (CMCase) is observed within five days of fermentation. A set of predicted results are obtained by analysis from computer software. The results shown in Table 1 concluded that cellulose of 1.5%, sucrose of 1.0% and peptone of 0.5 showed high activity of cellulase production under the process condition in which the inoculum is 3%, pH is 5 and temperature is 30° C.

TABLE 1
Production of Cellulase With Different Amount of Compositions

| | Cellu- | | | Cellulase | Cellulase |
|---|---|---|---|---|---|
| | lose | Sucrose | Peptone | (CMcase), U/mL | (CMcase), U/mL |
| Run | (%) | (%) | (%) | (Experimental) | (Predicted) |
| 1 | 1.50 | 2.00 | 0.10 | 0.8 | 2.0 |
| 2 | 0.50 | 3.50 | 0.10 | 1.1 | 0.6 |
| 3 | 2.50 | 3.50 | 0.10 | 0.4 | 0.0 |
| 4 | 1.50 | 3.50 | 0.50 | 9.0 | 12.1 |
| 5 | 0.50 | 2.00 | 0.50 | 6.6 | 8.9 |
| 6 | 1.50 | 2.00 | 0.50 | 11.2 | 11.2 |
| 7 | 0.50 | 0.50 | 0.10 | 6.3 | 5.1 |
| 8 | 2.50 | 0.50 | 0.10 | 1.6 | 3.6 |
| 9 | 1.50 | 2.00 | 0.50 | 10.8 | 11.2 |
| 10 | 2.50 | 0.50 | 0.90 | 2.4 | 3.7 |
| 11 | 2.50 | 3.50 | 0.90 | 3.8 | 4.2 |
| 12 | 1.50 | 2.00 | 0.90 | 3.0 | 2.3 |
| 13 | 0.50 | 0.50 | 0.90 | 0.0 | 0.6 |
| 14 | 1.50 | 2.00 | 0.50 | 13.0 | 11.2 |
| 15 | 0.50 | 3.50 | 0.90 | 2.4 | 1.1 |
| 16 | 2.50 | 2.00 | 0.50 | 10.9 | 9.7 |
| 17 | 1.50 | 1.00 | 0.50 | 16.4 | 13.9 |

### Example 2

The aeration and agitation rates of the fermentation process in a 30 L bioreactor is found by using the optimum media as described in Example 1. These optimum values are used for pilot scale production of cellulase via fermentation in a 300 L bioreactor. The results from this study indicate that when the agitation rate is 100 rpm and aeration rate is 1.0 vvm, cellulase production is 20-22 U/mL, which is the highest value in this experiment.

### Example 3

The scale-up production of cellulase in 300 L of stirred tank bioreactor is set up based on the production of cellulase in a 30 L bioreactor by applying three major scale-up strategies as listed below. The values of the agitation and aeration rates for the fermentation in the 300 L bioreactor are calculated in accordance to the agitation and aeration rates of the fermentation conducted in the 30L bioreactor that is shown in Table 2.

Strategy 1: Power number and superficial velocity (based 1 vvm) are constant

N₂=(D₁/D₂)^{2/3}×N₁ (1)

Strategy 2: Constant tip speed and superficial velocity (based 1 vvm) are constant

N₂=(D₁/D₂)×N₁ (2)

Strategy 3: Froude number and superficial velocity (based 0.5 vvm) are constant

N2=(D1/D2)1/2×N1 (3)

Where

D₁ is the impeller diameter of the 30 L bioreactor;

D₂ is the impeller diameter of the 300 L bioreactor;

N1 is the agitation/aeration rate of the 30 L bioreactor;

N2 is the agitation/aeration rate of the 300 L bioreactor; and

Superficial velocity=volumetric flow rate/area of bioreactor vessel.

TABLE 2
Three scale-up strategies for the production
of cellulase in a 300 L bioreactor

| Scale-up Strategy | Parameters |
|---|---|
| Strategy 1 | Agitation rate: 60 rpm; aeration rate: 0.5 vvm |
| Strategy 2 | Agitation rate: 46 rpm; aeration rate: 0.5 vvm |
| Strategy 3 | Agitation rate: 36 rpm; aeration rate: 0.3 vvm |

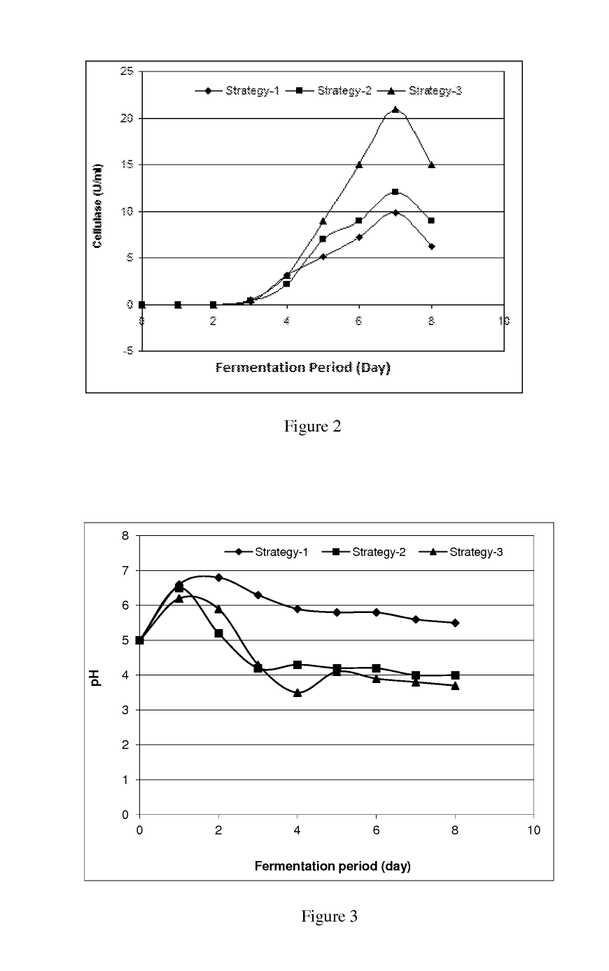

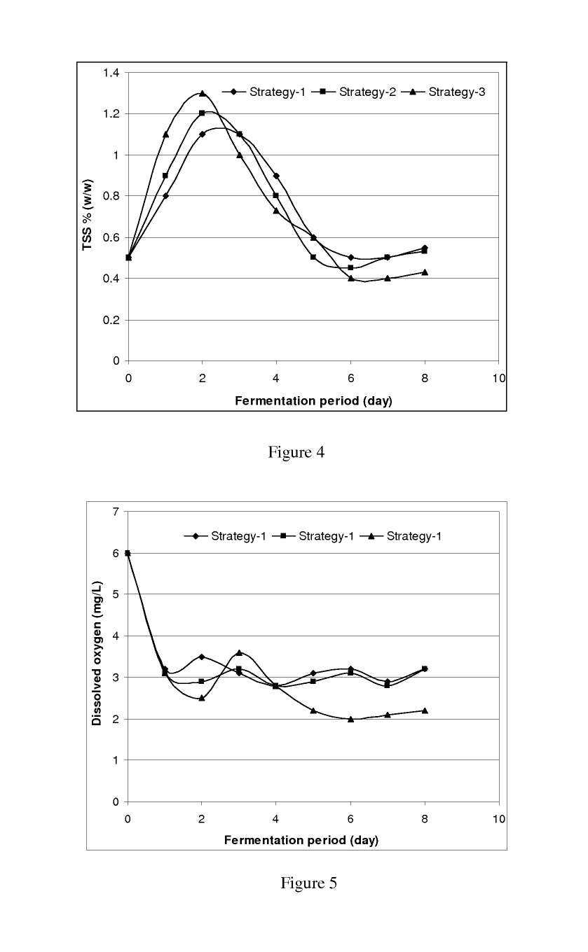

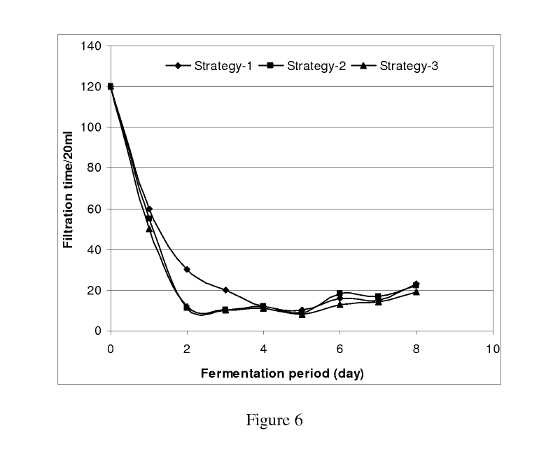

The optimum aeration rate of the production of cellulase from fermentation in the 30 L bioreactor is 0.5 to 1 vvm and the agitation rate is 100 rpm. The results of each scale-up strategy in terms of production of cellulase, pH, total suspended solids (TSS), dissolved oxygen, filterability are shown in FIGS. 2 to 6. The maximum production of cellulase obtained was 21 U/ml by Strategy 3. The maximum production of cellulase is found to occur when pH is 3 to 3.5. The results showed that the total suspended solids (TSS) content increased to 1% in 4 days of fermentation then decreased to 0.5% (w/w). The relationship of production of cellulase and fungal growth indicated that maximum production is achieved in the stationary and death phase of fungal growth. As shown in FIG. 6, the dissolved oxygen (DO) concentration or pO2 (%) is monitored throughout the fermentation process to evaluate the oxygen limitation in the growth and production. The results showed that the DO level is within 30 to 60% of pO2 indicating that the fermentation is carried out under aerobic condition for all the strategies. Filterability of fermentation broth is measured to evaluate the costing of down streaming process. The filterability was determined in filtration time (seconds) per 20 ml volume of filtrate. The results revealed that the time needed to filter 20 ml of filtrate is 10 to 30 seconds.

While the invention has been described with reference to an exemplary embodiment, it will be understood by those skilled in the art that various changes can be made and equivalents can be substituted for elements thereof without departing from the scope of the invention. In addition, many modifications can be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A method of producing cellulase, comprising:
performing fermentation using Trichoderma reesei in a medium containing sewage treatment plant sludge.

2. The method according to claim 1, wherein the medium further comprises a material selected from the group consisting of cellulose, sucrose, peptone, polysorbate 80, potassium dihydrogen phosphate, magnesium sulfate heptahydrate, and combinations thereof.

3. The method according to claim 1, further comprising stifling the medium at an agitation rate of 36 to 60 rpm.

4. The method according to claim 1, further comprising aerating the medium at 0.25 to 0.5 vvm.

5. The method according to claim 1, further comprising maintaining the medium at a temperature of 31 to 33° C. during fermentation.

6. The method according to claim 1, wherein the medium contains dissolved oxygen that has a concentration of more than 2 mg/L.
